(19) United States
(12) Patent Application Publication
(10) Pub. No.: US 2025/0177700 A1
(43) Pub. Date: Jun. 5, 2025

(54) DEVICE, SYSTEM, AND METHOD FOR DUAL PURPOSE TRACKING

(71) Applicant: FREENAV, INC., St. Paul, MN (US)

(72) Inventors: Brian L. Bechard, St. Paul, MN (US); Mina S. Fahim, New Brighton, MN (US); Greg A. Miller, Findlay, OH (US)

(21) Appl. No.: 18/967,893

(22) Filed: Dec. 4, 2024

Related U.S. Application Data
(60) Provisional application No. 63/605,718, filed on Dec. 4, 2023.

(51) Int. Cl. A61M 25/01; A61M 25/00

(52) U.S. Cl. CPC A61M 25/0127; A61M 25/005; A61M 25/0108; A61M 2025/0166; A61M 2205/0272; A61M 2205/3317; A61M 2205/6072

(57) ABSTRACT

A device includes a main body having a major portion formed from a ferromagnetic material configured for a dual purpose. The ferromagnetic material serves both a primary functioning purpose (structural, sensing, tracking, or therapeutic) and a secondary electromagnetic tracking purpose. The ferromagnetic material permits determining location and orientation of the device by a sensing system without requiring dedicated tracking sensors or manufacturing changes. The wireless unpowered ferromagnetic material generates trackable electromagnetic signatures when exposed to the magnetic fields, enabling precise location and orientation tracking while maintaining original device functionality without additional specialized sensors. This permits for the leveraging of existing ferromagnetic components like catheter braids, stent structures, or device frames that are already integrated into medical devices for their primary purposes.

### CROSS-REFERENCE TO RELATED APPLICATIONS

This application claims the benefit of U.S. Provisional Application No. 63/605,718, filed on Dec. 4, 2023. The entire disclosure of the above application is hereby incorporated herein by reference.

### FIELD

The present technology relates to a sensing system and method and associated device for tracking or determining the location and orientation of the device during a procedure such as, but not limited to, medical and surgical procedures, simulations, research, industrial applications, and supply chains and, more particularly, the location and orientation of devices such as medical instruments, catheters, and implants.

### INTRODUCTION

This section provides background information related to the present disclosure which is not necessarily prior art.

Various systems and methods are known in the art for tracking the location of an object in a variety of fields. In the medical field, specifically, it is often required to track the location of catheters, probes, endoscopic capsules and the like during medical procedures, in order to ascertain their location and orientation. Real-time location relative to other structures, for example, by overlaying location data onto pre-operative scan data is often beneficial to help guide procedures.

Previous approaches to tagging and identification systems have primarily focused on either optical or magnetic methods. Optical tags, such as reflective surfaces, barcodes, or QR codes, rely on visual scanning by cameras to retrieve information. These tags are limited in their ability to withstand harsh environments and may become inaccurate or illegible if damaged or dirty. On the other hand, magnetic tags, such as RFID tags, utilize radio frequency signals for identification. While magnetic tags offer durability and can be detected from a distance, they lack the ability to provide visual information.

One example of an optical system is described in U.S. Pat. No. 6,675,040 to Cosman, which describes a camera system operable in conjunction with data processors and graphic displays to provide tracking of instruments, objects, patients, and other devices in a surgical setting. The objective is primarily to provide the physician with a constructed view of the patient's anatomy in relationship to a variety of surgical instruments. The instrument “tags” comprise a wide variety of visually distinguishable markers associated with each of the instruments.

Commercial electromagnetic tracking methods typically either use a permanent magnet or a search coil within the patient. In the first example, the permanent magnet is located by an array of magnetic field sensors, external to the patient. The magnitude and angle of the magnetic field is used to infer the position of the permanent magnet. Such systems are limited by a requirement for a permanent magnet of significant volume in order to produce a measurable signal, susceptibility to external magnetic fields (such as the earth's field), and inability to track multiple targets.

In the second example, external transmitter coils produce time-varying (AC) magnetic fields, and an inductive pick-up coil (search coil) is located within the patient, in which a voltage is induced. Location is inferred by the (known) properties of the transmitted fields. Such systems also require an internal coil of significant volume, as well as requiring voltages (or data) to be retrieved from the search coil in order for real-time location to be possible. Operation frequency (and hence, signal strength) is limited by the need to avoid distortion in the transmitted fields.

Known tracking approaches that rely on dedicated electromagnetic tags and sensors also present certain limitations and challenges. The manufacturing and integration of such specialized electromagnetic tracking components is expensive, with complicated manufacturing processes that can present supply chain issues. Modifying existing medical devices and instruments to incorporate dedicated electromagnetic tracking sensors requires significant retooling of manufacturing lines and validation of new processes, increasing costs and potentially compromising the original device functionality. Current approaches also often require cables to connect tracking sensors to external hardware and additional equipment to produce electromagnetic fields, further complicating device design and manufacturing.

These limitations have created barriers to widespread adoption of electromagnetic tracking capabilities in medical devices, as manufacturers must choose between expensive redesigns of existing products or foregoing tracking functionality entirely. The need to integrate separate electromagnetic tracking components also constrains device size and shape options, limiting the use of tracking in smaller instruments like catheters where space is already at a premium. This challenge is particularly acute when trying to maintain existing device specifications while adding electromagnetic tracking capabilities that require dedicated sensors or tags to be incorporated into the manufacturing process.

None of these previous approaches has provided a comprehensive solution that combines the features needed for effective tracking, particularly in medical and industrial applications.

There is a continuing need for a comprehensive solution that leverages existing ferromagnetic materials and components already present in medical devices and instruments to serve a dual purpose. Desirably, such a system and method: improves tracking accuracy by utilizing the inherent electromagnetic properties of existing ferromagnetic components without requiring additional dedicated tracking sensors; minimizes or eliminates the need for cables by enabling wireless unpowered tracking through the natural harmonic and intermodulation responses of the ferromagnetic materials; minimizes additional equipment requirements by using the ferromagnetic materials already integrated into devices for their primary structural, sensing, or therapeutic purposes; reduces costs by eliminating separate tracking components since existing ferromagnetic elements like splines, braids, or frames can serve both their original function and provide tracking capability; maintains the original size and shape specifications of medical devices while adding tracking capabilities through materials already present in their construction; and enables precise tracking of location and orientation without requiring modifications to conventional manufacturing processes since the ferromagnetic materials are already integrated into the devices for their primary purposes.

### SUMMARY

In concordance with the instant disclosure, a comprehensive solution that leverages existing ferromagnetic materials and components already present in medical devices and instruments to serve a dual purpose, and which improves tracking accuracy by utilizing the inherent electromagnetic properties of existing ferromagnetic components without requiring additional dedicated tracking sensors; minimizes or eliminates the need for cables by enabling wireless unpowered tracking through the natural harmonic and intermodulation responses of the ferromagnetic materials; minimizes additional equipment requirements by using the ferromagnetic materials already integrated into devices for their primary structural, sensing, or therapeutic purposes; reduces costs by eliminating separate tracking components since existing ferromagnetic elements like splines, braids, or frames can serve both their original function and provide tracking capability; maintains the original size and shape specifications of medical devices while adding tracking capabilities through materials already present in their construction; and enables precise tracking of location and orientation without requiring modifications to conventional manufacturing processes since the ferromagnetic materials are already integrated into the devices for their primary purposes, has surprisingly been discovered.

The present technology includes articles of manufacture, systems, and processes that relate to sensing systems and methods for determining the location and orientation of a device during an operation therefore including, but not limited to, medical and surgical procedures. More specifically, the present technology involves a dual purpose device to be tracked such as, but not limited to, a medical instrument or implant, a system of tracking, and a method for tracking using the same.

In one embodiment, a device may include a main body having a major portion formed from a ferromagnetic material. The ferromagnetic material may be configured for a dual purpose. The first purpose may be a primary functioning purpose selected from several options. One option may be an original structural purpose, such as a woven braid within a wall structure of a catheter that provides both structural reinforcement and electromagnetic tracking capability, or a stent structure that provides both vascular support and electromagnetic tracking capability. Another option may be an original sensing purpose, such as a magnetic field sensor component that provides both magnetic field sensing functionality and electromagnetic tracking capability, or an inductive sensing element that provides both inductive measurement capability and electromagnetic tracking capability. A further option may be an original tracking purpose, such as reflective spheres on a tracking frame that provides both optical motion tracking capability and electromagnetic tracking capability, or a radio-opaque marker that provides both radiographic visualization and electromagnetic tracking capability. Yet another option may be a therapeutic purpose, such as a therapeutic implant structure that provides both therapeutic tissue support and electromagnetic tracking capability, or a drug-eluting component that provides both controlled therapeutic release and electromagnetic tracking capability. The second purpose may be a secondary tracking purpose including an electromagnetic tracked fiducial purpose. The second purpose may optionally also include an optical tracked fiducial purpose. The ferromagnetic material may permit determining a location and orientation of the device by a sensing system.

In another embodiment, a system for tracking location and orientation of a device may include the device described above and a sensing system. The sensing system may include an electromagnetic sensing device configured to track the device by electromagnetic means. The electromagnetic sensing device may include a plurality of coils arranged to generate one or more magnetic fields. At least some of the plurality of coils may be arranged to receive harmonics, intermodulation products, or time dependent variations of the one or more magnetic fields. These received signals may be used to determine the location and orientation of the device. The electromagnetic sensing device may comprise selection coils arranged to generate a spatially-varying DC magnetic field and interrogation coils arranged to generate one or more AC magnetic fields. The selection coils may form a planar array configured to generate a field-free point or field-free line that can be moved to search for location of the device.

In a further embodiment, a method for tracking the location and orientation of a device may include several steps. The first step may be providing the device described in the first embodiment. The second step may be providing a sensing system including an electromagnetic sensing device configured to track the device by electromagnetic means. The third step may be identifying the device using at least one of electromagnetic sensing by the system and optical sensing. The fourth step may be determining the location and orientation of the device using at least one of electromagnetic sensing by the system and optical sensing. The device may be a medical instrument selected from several options: a catheter having a ferromagnetic braid, an endoscope incorporating the ferromagnetic material, a surgical implant, a diagnostic instrument, a surgical navigation tool, or a robotic surgical component. The device may alternatively be used in non-medical applications such as supply chain tracking, robotic navigation, motion capture for gaming analysis, and athletic performance analysis.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1E are block diagrams illustrating a device, according to some embodiments of the present disclosure.

FIG. 2 is a block diagram illustrating a system, according to some embodiments of the present disclosure.

FIG. 3 is a flowchart illustrating a method, according to some embodiments of the present disclosure.

FIG. 4 is a flowchart illustrating a method, according to some embodiments of the present disclosure.

FIGS. 5 and 6 are FIGS. showing a sensing system in use for tracked location and orientation, according to some embodiments of the present disclosure.

FIGS. 7A and 7B are perspective views of an optical tracking frame with reflective spheres, including major portions having the ferromagnetic material, according to some embodiments of the disclosure.

FIG. 8 is a cross-sectional side elevational view of a reflective sphere of an optical tracking frame taken at section line A-A in FIG. 7A, with a major portion of ferromagnetic material disposed in an interior of the reflective sphere according to one embodiment of the disclosure.

FIGS. 9-15 are schematic views of a main body of a device including different amounts, shapes, and distributions of ferromagnetic material according to some embodiments of the disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology minimizes the need for dedicated tracking sensors or tags by utilizing the inherent properties of existing ferromagnetic components, such as catheter braids, stent structures, or device frames, as non-limiting examples, to permit determination of location and orientation through an electromagnetic sensing system. This dual-purposing approach improves upon existing systems by avoiding modifications to conventional manufacturing processes since the ferromagnetic materials are already integrated into the devices for their primary structural, sensing, or therapeutic purposes. The present technology further enables tracking through wireless unpowered features of these existing ferromagnetic components, which naturally generate harmonics or intermodulation products when exposed to an electromagnetic field, allowing for precise tracking of location and orientation without requiring additional specialized sensors or manufacturing steps. Furthermore, the technology improves manufacturing efficiency and reduces costs by eliminating the need for separate tracking components, since existing ferromagnetic elements like splines, braids, or frames can be dual purposed to serve both their original structural function and provide electromagnetic tracking capability. This approach maintains the original quality and functionality of medical devices while adding tracking capabilities through the inherent properties of materials already present in their construction.

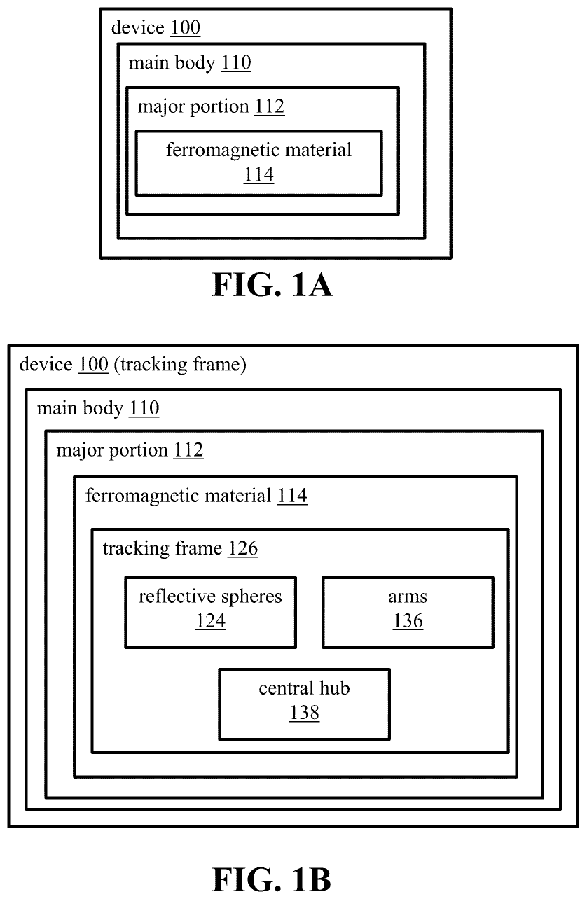

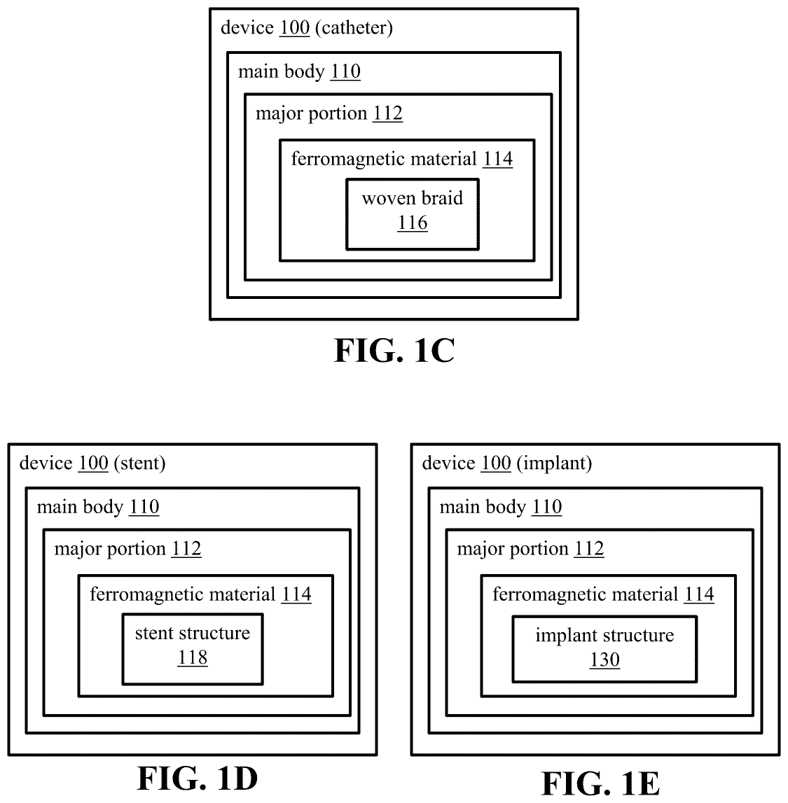

As shown in FIGS. 1A to 1E, a device 100 in accordance with the present disclosure may include a main body 110 with a major portion 112 formed from a ferromagnetic material 114. The ferromagnetic material 114 may be configured for a dual purpose including both a primary functioning purpose and a secondary tracking purpose. Without being bound to any particular theory, it is believed that the inherent presence of the ferromagnetic material 114 in the major portion 112 of the main body 110, as opposed to separate manufacture a later affixation of a tag to the main body 110, advantageously permits for a determining of a location and an orientation of the device by a sensing system 210 (shown in FIG. 2).

The sensing system 210 the present disclosure, as depicted in FIG. 2, for example, may be a system for determining the location and orientation of an object with an EM tag as described in U.S. Patent Application Ser. No. U.S. Pat. No. 20,200,060578 to Pooley, filed on Sep. 18, 2017, and titled “Sensing System and Method,” the entire disclosure of which is hereby incorporated herein by reference. Pooley describes a sensing system involving interrogation coils. At least some of the interrogation coils are arranged to generate one or more AC magnetic fields and at least some of the interrogation coils are arranged to receive harmonics, intermodulation products or time dependent variations of the AC magnetic fields, from which the orientation of an EM tag is determined in use. The sensing system in Pooley can also have selection coils, which may be arranged to generate a spatially-varying DC magnetic field from which the location of the tag can be determined in use.

With renewed attention to FIGS. 1A to 1E, the primary functioning purpose of the ferromagnetic material 114 may include at least one of an original structural purpose, an original sensing purpose, an original tracking purpose, a therapeutic purpose, and an original structural purpose. The secondary tracking purpose of the ferromagnetic material 114 may include at least one of an electromagnetic (EM) tracked fiducial purpose, and an optical tracked fiducial purpose, for example, as described further herein.

In one example, the original structural purpose of the ferromagnetic material includes forming the ferromagnetic material 114 into a woven braid 116 within a wall structure of a catheter, wherein the woven braid 116 may provide both structural reinforcement of the catheter and electromagnetic tracking capability. The ferromagnetic material 114 may also form a stent structure 118 that provides both vascular support and electromagnetic tracking capability. Other suitable original structural purposes may also be selected by one of ordinary skill in the art consistent with the present disclosure.

The primary functioning purpose may include the original sensing purpose, as shown in FIG. 1. The ferromagnetic material 114 may form a magnetic field sensor component that provides both magnetic field sensing functionality and electromagnetic tracking capability. Additionally, the ferromagnetic material 114 may form an inductive sensing element that provides both inductive measurement capability and electromagnetic tracking capability. Other suitable original sensing purposes may also be selected by one of ordinary skill in the art consistent with the present disclosure.

Referring still to FIGS. 1A to 1E, the primary functioning purpose may include the original tracking purpose. As particularly shown within FIG. 1B, the ferromagnetic material 114 may form reflective spheres 124 on a tracking frame 126 that provides both optical motion tracking capability and electromagnetic tracking capability. The ferromagnetic material 114 may also form a radio-opaque marker that provides both radiographic visualization and electromagnetic tracking capability. Other suitable original tracking purposes may also be selected by one of ordinary skill in the art consistent with the present disclosure.

The primary functioning purpose may include the therapeutic purpose, also as shown in FIG. 1. The ferromagnetic material 114 may form a therapeutic implant structure 130 that provides both therapeutic tissue support and electromagnetic tracking capability, such as show in FIG. 1E. Additionally, the ferromagnetic material 114 may form a drug-eluting component that provides both controlled therapeutic release and electromagnetic tracking capability. Other suitable therapeutic purposes may also be selected by one of ordinary skill in the art consistent with the present disclosure.

With reference to FIGS. 1A-E, the secondary tracking purpose may include an electromagnetic (EM) tracked fiducial purpose. The ferromagnetic material 114 may form a wireless unpowered feature within the major portion 112 that generates harmonics or intermodulation products when exposed to an electromagnetic field for tracking of the location and the orientation. The ferromagnetic material 114 of the major portion 112 may have a high aspect ratio quality that decreases self-demagnetization effects and increases effective magnetic permeability for electromagnetic tracking. Further, the ferromagnetic material 114 may have a high aspect ratio quality that decreases self-demagnetization effects and increases effective magnetic permeability. Also, the ferromagnetic material 114 may be configured to enter an inductor-capacitor resonance or magneto-mechanical resonance at specific AC field frequencies to produce unique electromagnetic signatures; or it may be arranged in a predetermined geometry to generate characteristic spectra and field strength dependencies for distinguishing between different tags. Other suitable electromagnetic (EM) tracked fiducial purposes may also be selected by one of ordinary skill in the art consistent with the present disclosure.

Where present, it should be appreciated that the optical tracked fiducial purpose may involve several options. For example, the ferromagnetic material may have either a two-dimensional or three-dimensional QR code pattern that provides both visual identification and electromagnetic tracking capabilities. The ferromagnetic material may also form reflective spheres that enable both camera tracking and electromagnetic field detection. The ferromagnetic material may have a surface relief pattern with a depth dimension creating both an optically scannable identifier and unique electromagnetic signature. The ferromagnetic material may further be distributed in a non-repeating pattern providing both visual reference points for optical tracking and electromagnetic tracking capabilities. In this manner, the non-repeating pattern may provide a plurality of individualized electromagnetic tracking signatures, for example. Other suitable optical tracked fiducial purposes may also be selected by one of ordinary skill in the art consistent with the present disclosure.

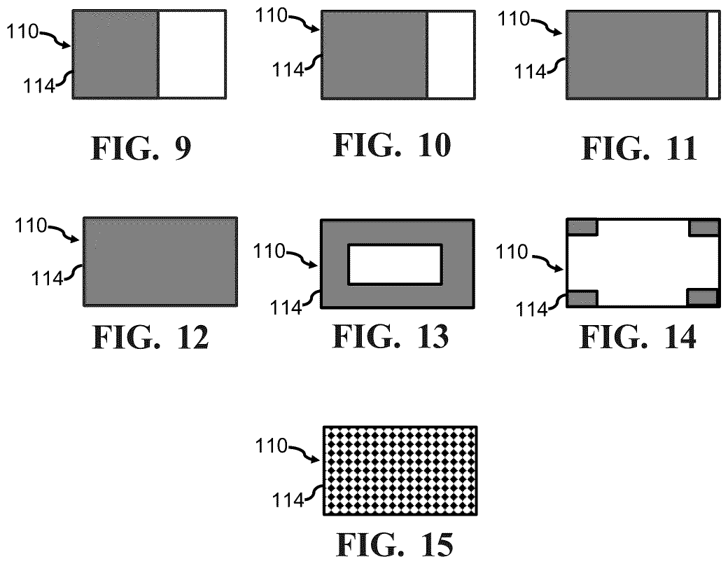

In certain embodiments, the amount of the ferromagnetic material 114 is significantly more than would be provided through the application of a conventional tag to the device 100. For example, the major portion 112 may be formed from the ferromagnetic material 114. In particular, the major portion 112 of the main body 110 may comprise at least 50% of the main body 110 by weight (FIG. 9), more particularly at least 70% of the main body 110 by weight (FIG. 10), and still more most particularly at least 90% of the main body 110 by weight (FIG. 11), and most particularly at least 100% of the main body 110 by weight (FIG. 12).

Additionally, the main body 110 may include any appropriately desired configuration of ferromagnetic material 114 including the front and the back of the major portion 112 of the main body 110. For example, such as shown in 13 where the ferromagnetic material 114 is formed on an outside and as shown in FIG. 14, where the ferromagnetic material 114 is shown as a coating and at corners of the main body 110. Additionally, the ferromagnetic material 114 may be formed of intentionally desired shapes, such as triangles or diamonds, as shown in FIG. 15.

The amount of ferromagnetic material 114 forming the major portion 112 may therefore be selected to be sufficient to permit a determining a location and an orientation of the device 100 by the sensing system 200 through the generation of harmonics or intermodulation products when exposed to an electromagnetic field. One of ordinary skill in the art may select a suitable amount of the ferromagnetic material 114 for the main body 110, within the scope of the present disclosure.

With continued reference to FIG. 1, the shape of the ferromagnetic material 114 may further have a high aspect ratio quality that decreases self-demagnetization effects and increases effective magnetic permeability for electromagnetic tracking, in a particular embodiment. The ferromagnetic material 114 may be configured to enter an inductor-capacitor resonance or magneto-mechanical resonance at specific AC field frequencies to produce unique electromagnetic signatures. The ferromagnetic material 114 may be arranged in a predetermined geometry to generate characteristic spectra and field strength dependencies that allow distinction between different tags.

Referring to FIG. 1, the ferromagnetic material 114 may have high magnetic permeability and low coercivity. The ferromagnetic material 114 may be an amorphous or nanocrystalline alloy selected from: Metglas 2826 MB, VITROVAC, and VITROPERM. The ferromagnetic material 114 may be formed in or on the main body 110 in a shape having an aspect ratio greater than 100:1 between short and long edges. A skilled artisan may also select other suitable types of the ferromagnetic material 114, as desired.

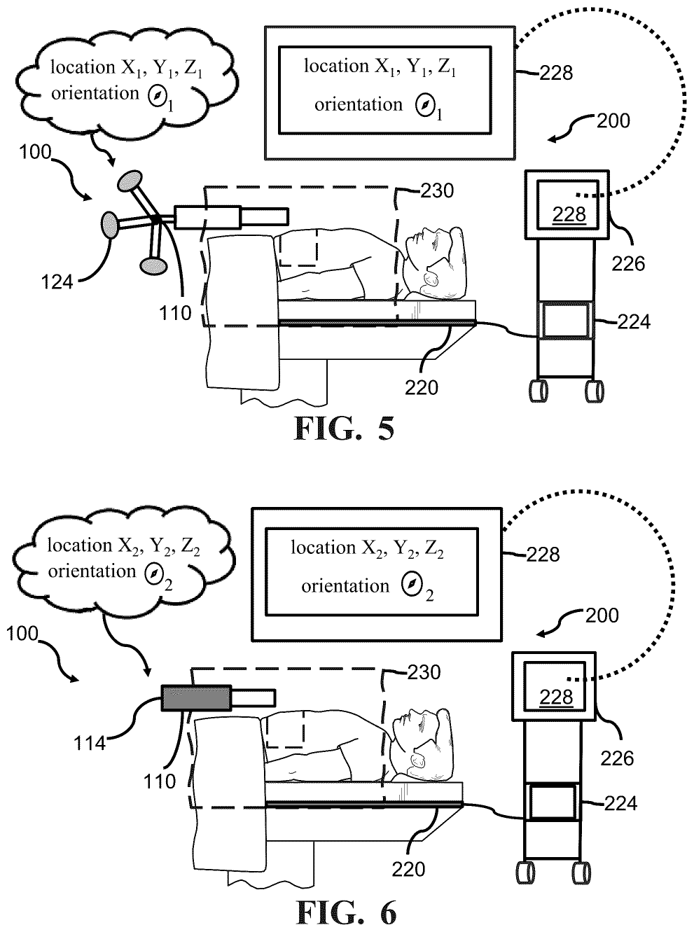

It should be appreciated that the device 100 of the present disclosure may be provided in a large variety of suitable forms. In one embodiment, for example, the device 100 may be configured as a tracking frame 126 (FIGS. 7A and 7B) where at least a portion of the tracking frame 126 is formed from the ferromagnetic material 114 to enable electromagnetic tracking capability. The tracking frame 126 may have multiple reflective spheres 124 arranged on it, for example, where each reflective sphere 124 has a ferromagnetic core and a reflective outer surface. The ferromagnetic core enables electromagnetic tracking while the reflective outer surface enables optical tracking, as described herein.

The reflective outer surface of the spheres 124 may be formed by either a reflective surface treatment or a reflective coating. The reflective coating may be wavelength-specific, for example, and most particularly an IR-reflective coating that is adapted for detection by an infrared camera. When applied to the ferromagnetic material, this coating enables both optical tracking via IR reflection and electromagnetic tracking via the ferromagnetic material.

The reflective spheres 124 may be configured to serve as reference points relative to one another for multiple purposes, including precision targeting, calibration, and imaging source registration. This configuration enables both optical and electromagnetic tracking simultaneously while providing multiple reference points for improved accuracy and reliability.

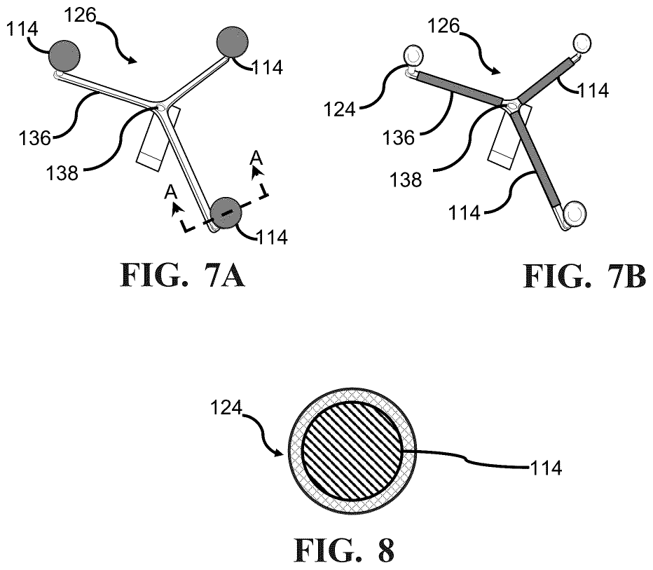

In a most particular embodiment, for example, illustrated in FIGS. 1B and in 7A-8, the tracking frame 126 may include a plurality of arms 136, and most particularly three arms 136. The three arms extend outwardly from a central hub 138, which many include a mounting structure including fasteners for mounting the tracking frame 126 to a medical instrument. Each of the three reflective spheres 124 may be mounted to a different one of the three arms 136. The reflective spheres 124 may be configured to serve as reference points, for example, relative to one another for precision targeting, calibration, and imaging source registration. In certain embodiments, such as shown within FIGS. 7A-7B and 8, one of a reflective sphere 124 and an arm 136 or the central hub 138 may include the ferromagnetic material 114. As shown in FIG. 8, in certain embodiments, the ferromagnetic material 114 may be an internal component of a reflective sphere 124. Other suitable constructions for the tracking frame are also contemplated and may be employed within the scope of the present disclosure, as desired.

In another example, the device 100 may be configured as a catheter to enable electromagnetic tracking during catheterization procedures. The catheter may be one of an EP catheter and a heart valve catheter enabling electromagnetic tracking during cardiac procedures. The ferromagnetic material 114 may form at least part of a woven braid and a spline component within a wall structure of the catheter that provides both structural support and electromagnetic tracking capability. Other suitable constructions for the catheter are also contemplated and may be employed within the scope of the present disclosure, as desired.

In a further embodiment, the device 100 may be configured as an endoscope to enable position and orientation tracking during endoscopic procedures. The ferromagnetic material 114 may be integrated into the endoscope structure to provide both mechanical support and electromagnetic tracking capability without requiring additional dedicated sensors. Other suitable constructions for the endoscope are also contemplated and may be employed within the scope of the present disclosure, as desired.

In yet another example, the device 100 may be configured as a stent to provide both vascular support and electromagnetic tracking capability during and after an implantation procedure. The ferromagnetic material 114 may be incorporated into the stent structure itself, enabling tracking while maintaining the primary therapeutic function. Other suitable constructions for the stent are also contemplated and may be employed within the scope of the present disclosure, as desired.

In yet a further example, the device 100 may be configured as a surgical fixture to enable real-time tracking relative to an anatomy of a patient during a surgical procedure. The ferromagnetic material 114 may be integrated into the fixture structure to provide both mechanical stability and electromagnetic tracking capability. For example, as shown within FIG. 6 where the ferromagnetic material 114 is integrated into the body 110 as appropriately desired. Other suitable constructions for the surgical fixture are also contemplated and may be employed within the scope of the present disclosure, as desired.

In an additional non-limiting example, the device 100 may be configured as an end effector of a robotic surgical limb to enable precise tracking of robotic movements during a robotic surgical procedure. The ferromagnetic material 114 may be incorporated into the end effector structure to provide both mechanical functionality and electromagnetic tracking capability. Other suitable constructions for the end effector are also contemplated and may be employed within the scope of the present disclosure, as desired.

In another non-limiting example, the device 100 may be configured as a diagnostic instrument to enable registration with imaging systems and precision targeting of anatomy. The diagnostic instrument configurations may include: a magnetic field sensing device incorporating the ferromagnetic material 114 as both a sensing component and tracking element; an inductive measurement device having ferromagnetic sensing elements that enable both measurement and position tracking; and a medical imaging registration device incorporating ferromagnetic fiducials for alignment with pre-operative scan data. Other suitable constructions for the diagnostic instrument are also contemplated and may be employed within the scope of the present disclosure, as desired.

In a further non-limiting example, the device 100 may be configured as a diagnostic capsule incorporating the ferromagnetic material 114 for tracking through a patient body; a precision targeting instrument having ferromagnetic components for real-time location verification; a diagnostic probe such as an ultrasound probe incorporating the ferromagnetic material 114 for position and orientation sensing; a medical scanning device having ferromagnetic reference markers for spatial registration; and a diagnostic equipment calibration device incorporating the ferromagnetic material 114 for spatial alignment verification. Other suitable constructions for the diagnostic capsule are also contemplated and may be employed within the scope of the present disclosure, as desired.

In yet another non-limiting example, the device 100 may be configured as a medical implant that provides both therapeutic tissue support and electromagnetic tracking capability. The medical implant configurations may include: a vascular stent configured to provide both structural support and electromagnetic tracking capability; a heart valve configured for electromagnetic tracking during and after implantation; and an orthopedic implant having ferromagnetic components for position tracking. Other suitable constructions for the medical implant are also contemplated and may be employed within the scope of the present disclosure, as desired.

In an additional non-limiting example, the device 100 may be configured as a therapeutic tissue support implant incorporating the ferromagnetic material 114; a drug-eluting implant incorporating the ferromagnetic material 114 for tracking drug delivery location; a surgical mesh incorporating woven ferromagnetic fibers; and an implantable medical device having ferromagnetic components for post-implantation location verification. Other suitable constructions for the therapeutic tissue support are also contemplated and may be employed within the scope of the present disclosure, as desired.

In another non-limiting example, the device 100 may be configured as a medical or surgical instrument to enable real-time tracking relative to other tracked devices in an operating arena. The surgical instrument configurations may include: a catheter having a woven braid formed from the ferromagnetic material 114; an endoscope incorporating the ferromagnetic material 114 for position and orientation tracking; and a surgical fixture having ferromagnetic components for real-time tracking relative to patient anatomy. Other suitable constructions for the medical or surgical instrument are also contemplated and may be employed within the scope of the present disclosure, as desired.

In a further non-limiting example, the device 100 may be configured as a robotic surgical limb incorporating the ferromagnetic material 114 for precise movement tracking; a surgical navigation instrument having ferromagnetic markers for registration with imaging systems; a diagnostic instrument incorporating the ferromagnetic material 114 for precision targeting; a surgical tool having ferromagnetic components for tracking relative to other instruments in an operating arena; and an operating suite bed or CT scanner bed incorporating ferromagnetic reference markers. Other suitable constructions for the robotic surgical limb are also contemplated and may be employed within the scope of the present disclosure, as desired.

Although the various configurations of the device 100 described hereinabove are believed to especially usable in accordance with the present disclosure, it should be understood that other suitable configurations including combinations of the various configurations described are contemplated and one of ordinary skill in the art may select other such suitable configurations consistent with the teachings of the present disclosure, as desired.

Referring now to FIG. 2, the system 200 for tracking location and orientation may include the device 100 and a sensing system 210 having an electromagnetic sensing device 212. The sensing system 210 may be provided as the system for determining the location and orientation of an object with an EM tag described in U.S. Patent Application Serial No. US20200060578 to Pooley, filed on Sep. 18, 2017, and titled “Sensing System and Method,” the entire disclosure of which is hereby incorporated herein by reference. The electromagnetic sensing device 212 may include a plurality of coils 214 arranged to generate one or more magnetic fields. At least some of the plurality of coils 214 may be arranged to receive harmonics, intermodulation products, or time dependent variations of the magnetic fields, from which the location and orientation of the device 100 may be determined.

With continued reference to FIG. 2, the electromagnetic sensing device 212 may include selection coils 216 arranged to generate a spatially-varying DC magnetic field and interrogation coils 218 arranged to generate one or more AC magnetic fields. The selection coils 216 may form a planar array configured to generate a field-free point or field-free line that can be moved to search for location of the device 100.

In one non-limiting example of the system 200, the electromagnetic sensing device 212 may be configured to determine the device 100 location based on received harmonics, intermodulation products or time dependent variations of the AC magnetic fields, for example, when a relevant component of the DC magnetic field at the device location is close to zero.

In another non-limiting example of the system 200, the sensing system 210 may be arranged to separate generation and reception of AC magnetic fields in time using a pulse-echo drive scheme. This configuration may enable improved signal detection and reduced interference during tracking operations.

In a further non-limiting example of the system 200, multiple devices 100 may be tracked simultaneously through the unique electromagnetic signatures generated by their respective ferromagnetic material 114. The tracking may be accomplished through the distinct harmonic responses and field dependencies of different ferromagnetic components.

Although the determination of the location and orientation of the device 100 is described hereinabove primarily with respect to the sensing system by Pooley, it should be understood that other suitable sensing systems are contemplated for use with the device 100 of the present disclosure. Likewise, one of ordinary skill in the art may select other such suitable sensing systems consistent with the teachings of the present disclosure, as desired.

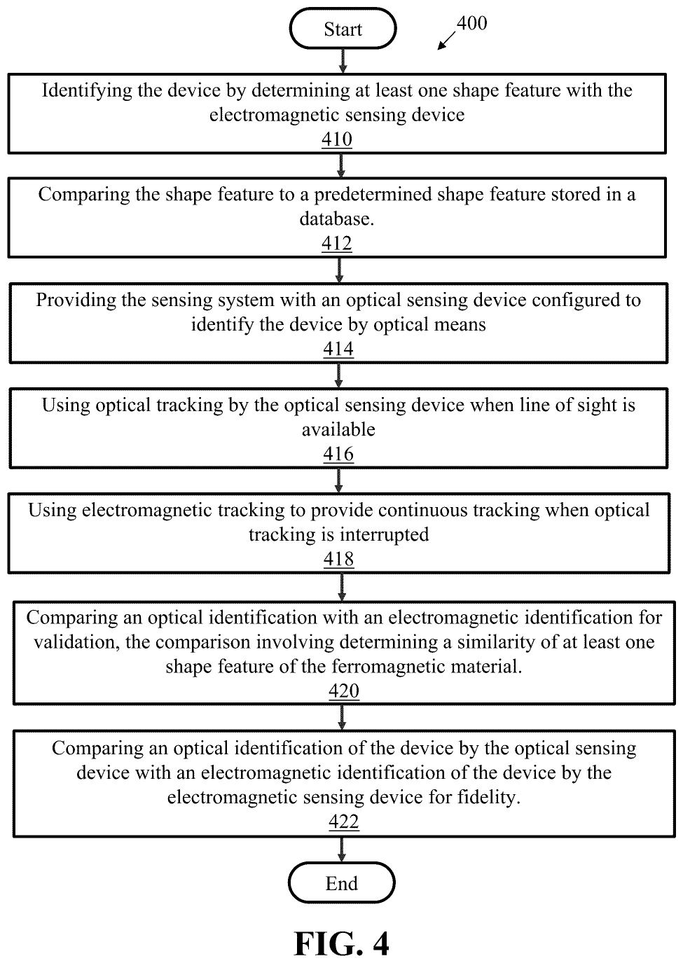

In particular, such as shown within FIGS. 5 and 6, the system 200 may be integrated with an EM field generator 220 for generating an electromagnetic field 230, a position tracking interface 224, and a computing device 226 for controlling an operation of the system 200 through one or more executable instructions stored in a memory of the computing device 226.

For example, the EM field generator 220 may be configured for determination of a location and an orientation of the device 100 such as according to the methods described in U.S. Patent Application Serial No. US20200060578 to Pooley. The position tracking interface 224 may be configured for transmitting the EM field generator 220 and sending real-time location and orientation data of sensors such as the device 100 including the ferromagnetic material 114. As further shown in FIGS. 5 and 6, a location and orientation or poise of the device 100 may thereby be displayed on screen 228 of the system 200 in a manner that is consistent with the actual location and orientation of the device 100 in operation.

As shown within FIG. 5, the device 100 may include a medical instrument having mounted thereto a tracking device including, for example, the reflective spheres 124 with the ferromagnetic material 114 and the tracking frame 126 mounted at the hub to the medical instrument, such as described above.

Alternatively, as shown within FIG. 6, the major portion 112 of the main body 110 of the device 100 instead includes the ferromagnetic material 114 in a sufficient amount that renders it functionally unnecessary to mount the separate tracking device to the medical instrument. In this manner, it should be appreciated that the present disclosure can permit for an elimination of tracking devices in the form of both small tags and also larger mounted tracking components, both of which can be difficult to place or use in operation.

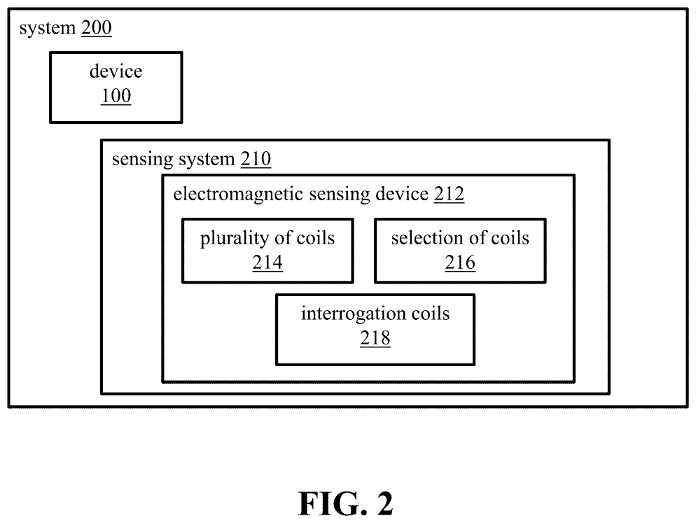

Referring now to FIG. 3, a method 300 for tracking location an including the reed orientation may include: a first step 302 of providing the device 100, such as described above; a second step 304 of providing the sensing system 200; a third step 306 of identifying the device 100 using electromagnetic sensing; and a fourth step 308 of determining the location and orientation of the device 100 using at least one of electromagnetic sensing and optical sensing. The method 300 of FIG. 3, as illustrated, may particularly be used without requiring separate tags or the use of mounted tracking components on instrumentation, and instead can take advantage of the ferromagnetic materials used in a dual purpose way with the instrumentation itself as described hereinabove.

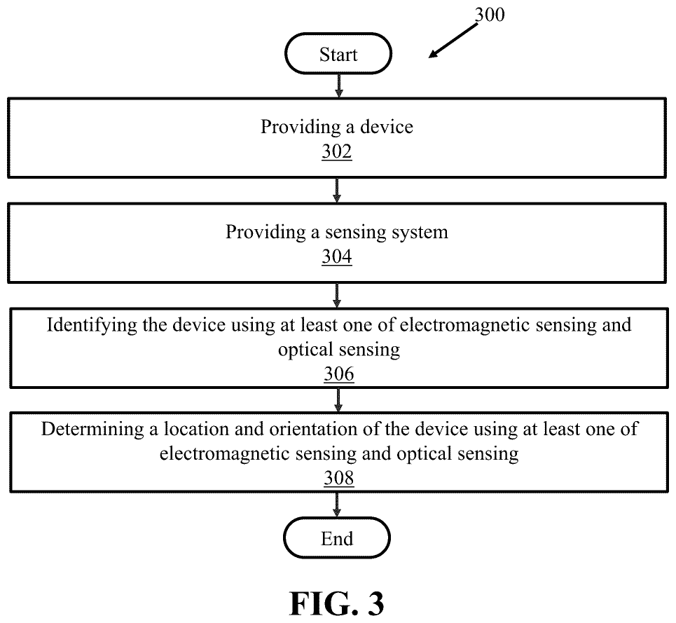

FIG. 4 is a flowchart that further describes another method 400, which can be performed as a continuation of the method 300 shown in FIG. 3, according to some embodiments of the present disclosure. In some embodiments, the method 400 may include additional steps of: a fifth step 410 of identifying the device 100 by determining at least one shape feature with the electromagnetic sensing device 212; and a sixth step 412 of comparing the shape feature to a predetermined shape feature stored in a database. The shape feature may be at least one of a configuration, volume, thickness, and geometry of the ferromagnetic material 114, for example. Other suitable types of the shape feature may also be selected for use by a skilled artisan, as desired, and within the scope of the present disclosure.

As illustrated in FIG. 4, the method 400 may also include: a seventh step 414 of providing the sensing system with an optical sensing device configured to identify the device by optical means; and an eighth step 416 of using optical tracking by the optical sensing device when line of sight is available. With continued reference to FIG. 4, the method 400 may further include: a ninth step 418 of using electromagnetic tracking to provide continuous tracking when optical tracking is interrupted; a tenth step 420 of comparing an optical identification with an electromagnetic identification for validation, the comparison involving determining a similarity of at least one shape feature of the ferromagnetic material 114; and a eleventh step 422 of comparing an optical identification of the device 100 by the optical sensing device with an electromagnetic identification of the device by the electromagnetic sensing device 212 for fidelity.

In one non-limiting example of either the method 300 or the method 400, the device 100 may be used in medical applications including: a catheter having a ferromagnetic braid; an endoscope incorporating the ferromagnetic material; a surgical implant; a diagnostic instrument; a surgical navigation tool; and a robotic surgical component.

In another non-limiting example of either the method 300 or the method 400, and the device 100 may be used in non-medical applications such as supply chain tracking, robotic navigation, motion capture for gaming analysis, and athletic performance analysis.

In a further non-limiting example of either the method 300 or the method 400, validation steps may be included to ensure accurate tracking. These steps may include comparing electromagnetic tracking data with known device geometries and configurations stored in a database. The validation process may help confirm proper device identification and tracking accuracy.

In yet another non-limiting example of either the method 300 or the method 400, compensation steps may be included to account for environmental factors that could affect tracking accuracy. These factors may include nearby ferromagnetic objects, electromagnetic interference, and variations in ambient magnetic fields. The compensation may be achieved through calibration and signal processing techniques.

Advantageously, the dual-purpose nature of the ferromagnetic material minimizes or eliminates the need for dedicated tracking components by enabling existing structural or functional elements to serve as tracking fiducials. For example, the ferromagnetic material may form a woven braid within a wall structure of a catheter that provides both structural reinforcement and electromagnetic tracking capability, or may form a stent structure that provides both vascular support and electromagnetic tracking capability.

The ferromagnetic material may also form existing components such as magnetic field sensor components, inductive sensing elements, reflective spheres, radio-opaque markers, therapeutic implant structures, or drug-eluting components-all of which can maintain their primary purpose while adding electromagnetic tracking functionality.

This dual-purpose approach significantly reduces device complexity and size in several ways. First, it eliminates the need for additional dedicated “tags” for EM tracking when the object being tracked already includes components and/or assemblies made of ferromagnetic material.

Second, it allows the ferromagnetic material to be integrated directly into existing device structures like catheter braids, stent frameworks, or surgical instrument components without requiring separate tracking elements. Third, it enables the use of smaller tracking elements since the ferromagnetic material can be incorporated into the device's existing structure rather than added as a separate component.

The system further addresses multiple limitations of prior art tracking systems. It improves tracking accuracy that was previously decreased by magnetic field distortion from other ferromagnetic devices in the environment.

It minimizes or eliminates the need for cables to connect tracking sensors to external hardware. It reduces the additional equipment required to produce an EM field for tracking. The system also minimizes costs associated with expensive sensors that are complicated to manufacture and can present supply chain issues. Additionally, it does not limit the size and shape of sensors, which previously restricted the use of known sensors and prevented their use with smaller catheters or surgical instruments. Finally, it removes the requirement for line-of-sight with cameras mounted a short distance from optical markers, providing greater flexibility in tracking applications.

### EXAMPLES

Example embodiments of the present technology are provided with reference to the several figures enclosed herewith. Although the present disclosure is described largely herein and below with respect to the field of medical technology, it should also be understood that applications of the present disclosure are not limited to the medical field and that other non- medical applications are contemplated and considered to be within the scope of the present disclosure.

### Example 1: Ultrasound Probe with Integrated EM Tracking

An ultrasound probe includes a main body formed substantially from a ferromagnetic material that provides both structural support and electromagnetic tracking capability. The ferromagnetic material comprises approximately 85% by weight of the main body, providing mechanical strength while enabling accurate position and orientation tracking. The material has high magnetic permeability and low coercivity, being formed from an amorphous or nanocrystalline alloy such as Metglas 2826MB or VITROVAC.

During use, the probe's position and orientation are tracked in real-time using the electromagnetic sensing system. The ferromagnetic material generates harmonics and intermodulation products when exposed to the system's magnetic fields. The selection coils generate a spatially-varying DC magnetic field while the interrogation coils produce AC fields, allowing precise determination of the probe's location and orientation relative to the patient.

The integrated tracking capability enables improved workflow during diagnostic procedures by allowing automatic registration between ultrasound images and other imaging modalities. The ferromagnetic construction eliminates the need for separate tracking sensors while maintaining the probe's ergonomic design and durability. The system achieves sub-millimeter tracking accuracy without requiring line-of-sight to external cameras.

### Example 2: EP Catheter with Ferromagnetic Construction

An electrophysiology (EP) catheter incorporates ferromagnetic material as the primary structural component of its wall structure. The ferromagnetic material comprises approximately 95% of the catheter body by weight, providing both mechanical support and electromagnetic tracking capability. The material is formed in a shape having an aspect ratio greater than 100:1 between short and long edges to enhance tracking performance.

The catheter's position and orientation are tracked during cardiac procedures using the electromagnetic sensing system. The ferromagnetic material generates distinct electromagnetic signatures when exposed to the system's AC and DC fields. The selection coils create a field-free point that can be moved throughout the tracking volume to precisely locate the catheter, while the interrogation coils enable determination of its orientation.

The integrated tracking capability allows real-time visualization of the catheter's position relative to cardiac anatomy, improving procedure accuracy and reducing fluoroscopy usage. The ferromagnetic construction eliminates the need for dedicated tracking sensors, enabling a smaller catheter profile while maintaining mechanical performance.

### Example 3: Surgical Navigation Instrument

A surgical navigation instrument is constructed primarily from ferromagnetic material that serves as both the structural framework and tracking medium. The ferromagnetic material comprises approximately 75% by weight of the instrument body and is formed from a high-permeability, low-coercivity alloy that enables both position and orientation tracking.

During surgical procedures, the instrument's position and orientation are continuously tracked using the electromagnetic sensing system. The ferromagnetic material generates characteristic harmonics and intermodulation products when exposed to the system's magnetic fields. The selection coils create field-free points or lines that can be swept through the tracking volume to locate the instrument.

The ferromagnetic construction enables precise navigation during minimally invasive procedures by providing real-time position and orientation data that can be overlaid on pre-operative imaging. The integration of tracking capability into the instrument's structure eliminates the need for external tracking arrays while maintaining functionality and ergonomics.

### Example 4: Robotic Navigation System

A robotic end effector is constructed primarily from ferromagnetic material that provides both structural integrity and electromagnetic tracking capability. The ferromagnetic material comprises approximately 85% of the end effector's body by weight and is formed into a shape that optimizes both mechanical strength and tracking performance.

The system tracks the position and orientation of the end effector using an array of electromagnetic sensors installed throughout the workspace. The ferromagnetic material generates unique electromagnetic signatures when exposed to the system's magnetic fields, allowing precise localization. The selection coils create field-free regions that can be moved to scan large volumes, while the interrogation coils enable determination of orientation.

The ferromagnetic construction enables automated navigation and real-time location tracking without requiring line-of-sight to optical sensors. The system achieves reliable tracking even in metallic environments typical of industrial settings while maintaining the end effector's mechanical capabilities.

### Example 5: Motion Capture System

A motion capture system incorporates performance capture equipment constructed primarily from ferromagnetic material. The equipment's main body is formed from high-permeability ferromagnetic material comprising approximately 90% by weight, serving as both the structural framework and electromagnetic tracking medium.

During capture sessions, the system tracks the position and orientation of multiple pieces of equipment simultaneously to record complex movements and interactions. The ferromagnetic material generates characteristic electromagnetic signatures through harmonics and intermodulation products when exposed to the system's magnetic fields. The selection coils create field-free points that can be rapidly moved throughout the capture volume.

The ferromagnetic construction provides several advantages over traditional optical motion capture systems. It eliminates the need for line-of-sight between markers and cameras, allowing capture of complex movements even when equipment is occluded. The system achieves reliable tracking in varying lighting conditions and metallic environments typical of production stages.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A device, comprising:
a main body having a major portion that is formed from a ferromagnetic material, the ferromagnetic material configured for a dual purpose including
a primary functioning purpose, including at least one of
an original structural purpose,
an original sensing purpose,
an original tracking purpose, and
a therapeutic purpose; and
a secondary tracking purpose, including
an electromagnetic (EM) tracked fiducial purpose,
wherein the ferromagnetic material permits for a determining of a location and an orientation of the device by a sensing system.

2. The device of claim 1, wherein the major portion is at least fifty percent (50%) of the main body by weight.

3. The device of claim 1, wherein the original structural purpose includes one of:
the ferromagnetic material forming a woven braid within a wall structure of a catheter, wherein the woven braid provides both structural reinforcement of the catheter and electromagnetic tracking capability; and
the ferromagnetic material forming a stent structure that provides both vascular support and electromagnetic tracking capability.

4. The device of claim 1, wherein the original sensing purpose includes one of:
the ferromagnetic material forming a magnetic field sensor component that provides both magnetic field sensing functionality and electromagnetic tracking capability; and
the ferromagnetic material forming an inductive sensing element that provides both inductive measurement capability and electromagnetic tracking capability.

5. The device of claim 1, wherein the original tracking purpose includes one of:
the ferromagnetic material forming reflective spheres on a tracking frame that provides both optical motion tracking capability and electromagnetic tracking capability; and
the ferromagnetic material forming a radio-opaque marker that provides both radiographic visualization and electromagnetic tracking capability.

6. The device of claim 1, wherein the therapeutic purpose includes one of
the ferromagnetic material forming a therapeutic implant structure that provides both therapeutic tissue support and electromagnetic tracking capability; and
the ferromagnetic material forming a drug-eluting component that provides both controlled therapeutic release and electromagnetic tracking capability.

7. The device of claim 1, wherein the electromagnetic (EM) tracked fiducial purpose includes one of
the ferromagnetic material forming a wireless unpowered feature within the major portion that generates harmonics or intermodulation products when exposed to an electromagnetic field for tracking of the location and the orientation;
the ferromagnetic material of the major portion having a high aspect ratio quality that decreases self-demagnetization effects and increases effective magnetic permeability for electromagnetic tracking;
the ferromagnetic material of the major portion being configured to enter an inductor-capacitor resonance or magneto-mechanical resonance at specific AC field frequencies to produce unique electromagnetic signatures; and
the ferromagnetic material of the major portion being arranged in a predetermined geometry to generate characteristic spectra and field strength dependencies that allow distinction between different tags.

8. The device of claim 1, wherein the secondary tracking purpose further includes an optical tracked fiducial purpose including one of:
the ferromagnetic material having either a two-dimensional QR code pattern or a three-dimensional QR code pattern that provides both visual identification and electromagnetic tracking capabilities;
the ferromagnetic material forming reflective spheres that enable both camera tracking and electromagnetic field detection;
the ferromagnetic material having a surface relief pattern with a depth dimension that creates both an optically scannable identifier and a unique electromagnetic signature; and
the ferromagnetic material being distributed in a non-repeating pattern that provides both visual reference points for optical tracking and electromagnetic tracking capabilities.

9. The device of claim 1, wherein the main body includes a tracking frame, and at least a portion of the tracking frame is formed from the ferromagnetic material.

10. The device of claim 1, wherein the main body includes a tracking frame and a plurality of reflective spheres arranged on the tracking frame, and each of the plurality of reflective spheres has a ferromagnetic core and a reflective outer surface, the ferromagnetic core enabling the EM tracking while the reflective outer surface enabling optical tracking.

11. The device of claim 10, wherein the reflective outer surface is formed by at least one of a reflective surface treatment and a reflective coating.

12. A system for tracking location and orientation of an object, comprising:
a device including a main body having a major portion that is formed from a ferromagnetic material, the ferromagnetic material configured for a dual purpose including
a primary functioning purpose, including at least one of
an original structural purpose,
an original sensing purpose,
an original tracking purpose, and
a therapeutic purpose; and
a secondary tracking purpose, including
an electromagnetic (EM) tracked fiducial purpose; and
a sensing system including an electromagnetic sensing device configured to track the device by electromagnetic means, wherein the ferromagnetic material permits for a determining of a location and an orientation of the device by the sensing system.

13. The system of claim 12, wherein the electromagnetic sensing device includes a plurality of coils arranged to generate one or more magnetic fields, and at least some of the plurality of coils arranged to receive harmonics, intermodulation products, or time dependent variations of the one or more magnetic fields, from which the location and the orientation of the device is determined.

14. A method for tracking location and orientation of an object, comprising steps of:
providing a device including a main body having a major portion that is formed from a ferromagnetic material, the ferromagnetic material configured for a dual purpose including
a primary functioning purpose, including at least one of
an original structural purpose,
an original sensing purpose,
an original tracking purpose, and
a therapeutic purpose; and
a secondary tracking purpose, including
an electromagnetic (EM) tracked fiducial purpose;
providing a sensing system including an electromagnetic sensing device configured to track the device by electromagnetic means, wherein the ferromagnetic material permits for a determining of a location and an orientation of the device by the sensing system;
identifying the device using at least one of electromagnetic sensing by the sensing system and optical sensing; and
determining the location and the orientation of the device using at least one of electromagnetic sensing by the sensing system and optical sensing.

15. The method of claim 14, further comprising steps of:
identifying the device by determining at least one shape feature of the device with the electromagnetic sensing device; and
comparing the at least one shape feature to a predetermined shape feature stored in a database.

16. The method of claim 15, wherein the at least one shape feature is at least one of a configuration, a volume, a thickness, and a geometry of the ferromagnetic material of the device.

17. The method of claim 14, wherein the step of identifying the device further includes steps of:
providing the sensing system with an optical sensing device configured to identify the device by optical means.

18. The method of claim 17, further comprising steps of:
using optical tracking by the optical sensing device when line of sight is available; and
using electromagnetic tracking to provide continuous tracking when optical tracking is interrupted.

19. The method of claim 17, wherein the step of identifying the device further includes a step of comparing an optical identification with an electromagnetic identification for validation, the comparing involving a determining of a similarity of at least one shape feature of the ferromagnetic material.

20. The method of claim 17, further comprising comparing an optical identification of the device by the optical sensing device with an electromagnetic identification of the device by the electromagnetic sensing device for fidelity.
